(19) United States
(12) Patent Application Publication
(10) Pub. No.: US 2018/0153791 A1
(43) Pub. Date: Jun. 7, 2018

(54) COSMETIC AND PERSONAL CARE FORMULAS AND METHODS

(71) Applicant: Kobo Products, Inc., South Plainfield, NJ (US)

(72) Inventors: Stacey House, Edison, NJ (US); Catherine Mercado, Edison, NJ (US); Deborah McCarthy, Toms River, NJ (US); David Schlossman, Short Hills, NJ (US)

(21) Appl. No.: 15/570,541

(22) PCT Filed: Apr. 29, 2016
(86) PCT No.: PCT/US16/30307

§ 371 Date: Oct. 30, 2017

Related U.S. Application Data
(60) Provisional application No. 62/272,546, filed on Dec. 29, 2015.
(60) Provisional application No. 62/155,233, filed on Apr. 30, 2015.

(51) Int. Cl. A61K 8/891; A61Q 1/06; A61Q 1/04; A61Q 1/10; A61Q 1/02

(52) U.S. Cl. CPC A61K 8/891; A61Q 1/06; A61Q 1/02; A61Q 1/10; A61Q 1/04

(57) ABSTRACT

Applications of low-viscosity phenyl trimethicones in cosmetic and personal care products, cosmetic and personal care formulas containing low-viscosity phenyl trimethicones with various improved properties over use Phenyl Trimethicone @ 20 cSt or @ 40 cSt, and preparation methods thereof are disclosed.

### CROSS-REFERENCE TO RELATED APPLICATIONS

This application claims priority under 35 U.S.C. § 119(e) to U.S. Provisional Patent Application Ser. No. 62/155,233, filed on Apr. 30, 2015, and No. 62/272,546, filed on Dec. 29, 2015, both of which are incorporated herein by reference in their entireties.

### FIELD OF THE INVENTION

The present invention relates to use of a low-viscosity phenyl trimethicone, such as Phenyl Trimethicone @ 10 cSt, in cosmetic and personal care formulas to improve various properties.

### BACKGROUND OF THE INVENTION

Phenyl trimethicone, in particular Phenyl Trimethicone @ 20 cSt or @ 40 cSt, has been used in cosmetic and personal care industries to impart gloss, softness, and shine, for example, in lip balms, lipsticks, and lipglosses. It has also been used in hair care, antiperspirant, and sunscreen formulations. However, use of a low-viscosity phenyl trimethicone, such as Phenyl Trimethicone @ 10 cSt, in these formulas has not been reported.

### SUMMARY OF THE INVENTION

The present invention provides application of a low-viscosity phenyl trimethicone in cosmetic and personal care formulations to attain various unexpected superior properties. For example, use of Phenyl Trimethicone @ 10 cSt to replace Phenyl Trimethicone @ 20 cSt or @40 cSt afforded higher gloss measurements for various cosmetic formulas. The advantages also include, but are not limited to, improved shine, longer lasting effects upon wear, improved transfer resistance and smudge resistance, better color development of the pigment in the finished formula, and more uniform product payoff, etc. These results are atypical of raw materials in cosmetic and personal care formulations.

Thus, in one aspect, the present invention provides a method of improving properties of a cosmetic or personal care product, such as gloss, comprising adding an amount of a low-viscosity phenyl trimethicone, such as Phenyl Trimethicone @ 10 cSt, into the cosmetic or personal care formula.

In another aspect, the present invention provides cosmetic or personal care formulas comprising a sufficient amount of a low-viscosity phenyl trimethicone, e.g., Phenyl Trimethicone @ 10 cSt, such that the formula has a noticeable beneficial effect, such as improved shine and gloss, wear resistance, transfer resistance, and/or smudge resistance, etc., as compared to use of Phenyl Trimethicone @ 20 cSt and/or @40 cSt in the formula.

In another aspect, the present invention provides a method of preparing a cosmetic or personal care formula, comprising mixing an amount of a low-viscosity phenyl trimethicone, such as Phenyl Trimethicone @ 10 cSt, with other ingredients of the formula.

The cosmetic or personal care formulas applicable to the present invention include, but are not limited to, those used for lips, nails, skin, eyelashes, eyebrows, or hair, etc.

Therefore, the present invention provides use of low-viscosity phenyl trimethicone, e.g., Phenyl Trimethicone @ 10 cSt, as an alternative to Phenyl Trimethicone @ 20 cSt and/or @40 cSt to achieve higher visible gloss, among other superior properties, in cosmetic or personal care formulations.

Other aspects or benefits of the present invention will be reflected in the following drawings, detailed description, and claims.

### BRIEF DESCRIPTION OF THE DRAWINGS

FIG. 1 illustrates the comparison results on gloss, wear resistance and transfer resistance of a lipstick formula containing Phenyl Trimethicone @ 10 cSt with a corresponding lipstick formula containing Phenyl Trimethicone @ 20 cSt applied on the lips of testing person 1.

FIG. 2 illustrates the comparison results on gloss, wear resistance and transfer resistance of a lipstick formula containing Phenyl Trimethicone @ 10 cSt with a corresponding lipstick formula containing Phenyl Trimethicone @ 20 cSt applied on the lips of testing person 2.

FIG. 3 illustrates the results from an in-vivo transfer resistance test comparing a lipstick formula containing Phenyl Trimethicone @ 10 cSt with a corresponding formula containing Phenyl Trimethicone @ 20 cSt (A: panelist 1; B: panelist 2; and C: panelist 4).

### DETAILED DESCRIPTION OF THE INVENTION

The present invention is based on a surprising discovery that use of Phenyl Trimethicone @ 10 cSt in certain cosmetic formulations could enhance gloss, among other properties, in comparison to use of Phenyl Trimethicone @ 20 cSt and/or 40 cSt. In particular, use of Phenyl Trimethicone @ 10 cSt to attain higher specular reflection gloss than use of Phenyl Trimethicone @ 20 cSt and/or 40 cSt has been discovered in the application of lipstick and lipgloss formulas.

Because the gloss property constitutes an important property in many cosmetic products, the present invention is applicable to a variety of such cosmetic products, including but not limited to products used on the lips, nails, skin, eyelashes, eyebrows, or hair. In general, the cosmetic or personal care formulation of the present invention may be used topically on a subject where gloss constitutes an important feature.

The advantages of the present invention also include, without limitation, improved shine, a longer lasting effect upon wear, improved transfer resistance and smudge resistance, better color development of the pigment in the finished formula, and more uniform product payoff.

Thus, in one aspect the present invention provides cosmetic or personal care compositions for topical use on a subject, which comprises a low-viscosity phenyl trimethicone composition, e.g., Phenyl Trimethicone @ 10 cSt, in a sufficient amount that a desirable level of gloss can be attained. The amount of phenyl trimethicone @ 10 cSt can be in the range of about 1% to about 50% by weight depending on the end use of the product. For example, when used in a lipstick, the amount of phenyl trimethicone @ 10 cSt can be in the range of about 5% to about 50% by weight; in a lipgloss, the amount can be in the range of about 1% to about 50% by weight; in eyeliners, the amount can be in the range of about 1% to about 50% by weight; in mascara, the amount can be in the range of about 1% to about 35% by weight; and in foundation, the amount can be in the range of about 1% to about 70%.

The composition according to the present invention may be in the form of a lip makeup product, such as lipsticks and lipgloss, or in the form of an eye makeup product, such as mascara and eyeliner.

In one embodiment, the cosmetic composition is a lipstick formulation containing phenyl trimethicone @ 10 cSt in the amount of about 5-50% by weight.

In another embodiment, the cosmetic composition is a lipstick formulation containing phenyl trimethicone @ 10 cSt in the amount of about 10-45% by weight.

In another embodiment, the cosmetic composition is a lipstick formulation containing phenyl trimethicone @ 10 cSt in the amount of about 15-40% by weight.

In another embodiment, the cosmetic composition is a lipstick formulation containing phenyl trimethicone @ 10 cSt in the amount of about 20-35% by weight.

In another embodiment, the cosmetic composition is a lipstick formulation containing phenyl trimethicone @ 10 cSt in the amount of about 30-35% by weight.

In another embodiment, the lipstick formulation is substantially described in Table 1.

In one embodiment, the cosmetic composition is a lipgloss formulation containing phenyl trimethicone @ 10 cSt in the amount of about 1-50% by weight.

In another embodiment, the cosmetic composition is a lipgloss formulation containing phenyl trimethicone @ 10 cSt in the amount of about 2-40% by weight.

In another embodiment, the cosmetic composition is a lipgloss formulation containing phenyl trimethicone @ 10 cSt in the amount of about 3-30% by weight.

In another embodiment, the cosmetic composition is a lipgloss formulation containing phenyl trimethicone @ 10 cSt in the amount of about 4-20% by weight.

In another embodiment, the cosmetic composition is a lipgloss formulation containing phenyl trimethicone @ 10 cSt in the amount of about 5-15%.

In another embodiment, the cosmetic composition is a lipgloss formulation containing phenyl trimethicone @ 10 cSt in the amount of about 10%.

In another embodiment, the foundation formulation is substantially described in Table 4.

In one embodiment, the cosmetic composition is a mascara formulation containing phenyl trimethicone @ 10 cSt in the amount of about 1-50% by weight.

In another embodiment, the cosmetic composition is a mascara formulation containing phenyl trimethicone @ 10 cSt in the amount of about 2-40% by weight.

In another embodiment, the cosmetic composition is a mascara formulation containing phenyl trimethicone @ 10 cSt in the amount of about 3-30% by weight.

In another embodiment, the cosmetic composition is a mascara formulation, comprising phenyl trimethicone @ 10 cSt in the amount of about 4-20% by weight.

In another embodiment, the cosmetic composition is a mascara formulation containing phenyl trimethicone @ 10 cSt in the amount of about 5-15%.

In another embodiment, the cosmetic composition is a mascara formulation containing phenyl trimethicone @ 10 cSt in the amount of about 10%.

In another embodiment, the mascara formulation is substantially described in Table 7.

In one embodiment, the cosmetic composition is an eyeliner formulation containing phenyl trimethicone @ 10 cSt in the amount of about 1-50% by weight.

In another embodiment, the cosmetic composition is an eyeliner formulation containing phenyl trimethicone @ 10 cSt in the amount of about 2-40% by weight.

In another embodiment, the cosmetic composition is an eyeliner formulation containing phenyl trimethicone @ 10 cSt in the amount of about 3-30% by weight.

In another embodiment, the cosmetic composition is an eyeliner formulation containing phenyl trimethicone @ 10 cSt in the amount of about 4-20% by weight.

In another embodiment, the cosmetic composition is an eyeliner formulation containing phenyl trimethicone @ 10 cSt in the amount of about 5-15%.

In another embodiment, the eyeliner formulation is substantially described in Table 10.

In one embodiment, the cosmetic composition is a foundation formulation.

In another embodiment, the cosmetic composition is a foundation formulation containing phenyl trimethicone @ 10 cSt in the amount of about 1-70% by weight.

In another embodiment, the cosmetic composition is a foundation formulation containing phenyl trimethicone @ 10 cSt in the amount of about 1-50% by weight.

In another embodiment, the cosmetic composition is a foundation formulation containing phenyl trimethicone @ 10 cSt in the amount of about 5-30% by weight.

In another embodiment, the foundation formulation is substantially described in Table 13.

In other embodiments, the cosmetic formulation in any one of the embodiments described herein further comprises one or more cosmetically acceptable fillers and/or one or more cosmetically acceptable pigments.

In another aspect, the present invention provides a method of preparing a cosmetic or personal care formulation, comprising including a low viscosity phenyl trimethicone as one of the ingredients of the formulation to enhance at least one property of the formulation selected from the group consisting of gloss, shine, lasting effect upon wear, transfer resistance, smudge resistance, color development of the pigment in finished formula, and uniform product payoff.

In one embodiment of this aspect, the low viscosity phenyl trimethicone has a viscosity below 20 cSt.

In another embodiment of this aspect, the low viscosity phenyl trimethicone has a viscosity in the range of 1 to 15 cSt.

In another embodiment of this aspect, the low viscosity phenyl trimethicone has a viscosity in the range of 5 to 12.5 cSt.

In another embodiment of this aspect, the low viscosity phenyl trimethicone is Phenyl Trimethicone @ 10 cSt.

In some embodiments of this aspect, the cosmetic or personal care formulation is a formulation used for lips, nails, skin, eyelashes, eyebrows, or hair.

In another embodiment of this aspect, the cosmetic or personal care formulation is a formulation selected from the group consisting of lipstick, lipgloss, eyeliner, masacra, and foundation.

Other embodiments of the present invention include those as substantially shown and described and any possible combinations of any two or more embodiments described herein.

The term “low viscosity” or the like, as used herein on phenyl trimethicone polymer composition, means a viscosity below 20 cSt, preferably below 18 cSt, more preferably equal to or below 15 cSt, for example in the range of 1 cSt to 15 cSt, and more preferably in the range of 5 cSt to 12.5 cSt.

The terms “formulation” and “formula” may be used interchangeably in the present application.

The term “about,” as used herein, means that a number can vary up to ±20%, preferably within ±10%, and more preferably within ±5%. When “about” is used in front of a range, it applies to both upper and lower limits of the range.

The term “a,” “an,” or “the,” as used herein, represents both singular and plural forms. In general, when either a singular or a plural form of a noun is used, it denotes both singular and plural forms of the noun.

The present invention of using Phenyl Trimethicone @ 10 cSt as compared to using Phenyl Trimethicone @ 20 cSt has been discovered in emulsions, hot pours, and anhydrous gels.

The advantageous use of a low-viscosity phenyl trimethicone, e.g., Phenyl Trimethicone @ 10 cSt, in cosmetic and personal care industries according to the present invention is demonstrated in the following illustrative, non-limiting examples, including results from the studies on the use of Phenyl Trimethicone @ 10 cSt compared to use of Phenyl Trimethicone @ 20 cSt or @ 40 cSt in various cosmetic or personal care formulations. Though only lipstick, lipgloss, eyeliner, mascara, and foundation formulas are used to illustrate applications of the present invention, the Examples do not in any way limit the broader use of the formulations or variants thereof.

### EXAMPLES

### General Methods and Materials

The specular reflection gloss of the Phenyl Trimethicone (PT) materials (@10, 20, and 40 cSt) was measured using the Glossmeter; so was the specular reflection gloss of lipstick, lipgloss and mascara formulas containing the different PT materials (@10, 20, and 40 cSt). As used herein, the CPF-3300@10 cSt material has a viscosity range of 5-12.5 cSt, the CPF-3300@20 cSt material has a viscosity range of 19.5-22.5 cSt, and the CPF-3300@40 cSt material has a viscosity range of 37.5-42.5 cSt.

Sample drawdowns were conducted on a BYK Black and White Opacity Chart using 4 mm drawdown bars. Micro Gloss Meter was calibrated before each use and had a measurement time of 0.5 seconds.

### Example 1

Three lipstick formulas containing Phenyl Trimethicone @ 10 cSt, Phenyl Trimethicone @ 20 cSt, and Phenyl Trimethicone @ 40 cSt with all other components kept the same, are described in Table 1, Table 2, and Table 3, respectively. They were prepared using the identical procedure as provided below.

TABLE 1
Lipstick Formula with Phenyl Trimethicone @ 10 cSt

| Part | Ingredient | Percent | INCI |
|---|---|---|---|
| 1 | CPF-3300@10 cSt | 32.2 | Phenyl Trimethicone |
| 1 | Ozokerite Wax | 7.00 | Ozokerite |
| 1 | Koboguard HRPC | 7.00 | Hydrogenated Polyclopentadiene |
| | | | (and) Polyethylene (and) |
| | | | Copernicia Cerifera Wax (and) |
| | | | Tocopherol |
| 1 | Salacos WO-6 | 6.00 | Dipentaerythrityl Tri- |
| | | | Polyhydroxystearate |
| 1 | Carnuba Wax | 5.00 | Carnuba Wax |
| 1 | Microcrystalline | 4.00 | Microcrystalline |
| | Wax | | |
| 1 | Synthetic Beeswax | 4.00 | Beeswax |
| 2 | Cosmol 43V | 10.00 | Polyglyceryl-2 Triisostearate |
| 2 | INBP45R7C | 11.8 | Red 7 Lake (and) Isononyl |
| | | | Isononanoate (and) Isopropyl |
| | | | Myristate (and) Stearalkonium |
| | | | Hectorite (and) Isopropyl Titanium |
| | | | Triisostearate (and) Propylene |
| | | | Carbonate (and) |
| | | | Polyhydroxystearic Acid |
| 2 | KTZ Aruban Coral | 5.00 | Mica (and) Titanium Dioxide (and) |
| | | | Iron Oxides (C. I. 77491) |
| 2 | Kobopearl Perpetual | 3.00 | Synthetic Fluorphlogopite (and) |
| | RedGold | | Silica (and) Titanium Dioxide |
| 2 | TNP50T7-ATB | 4.00 | C12-15 Alkyl Benzoate (And) |
| | | | Titanium Dioxide (And) Argania |
| | | | Spinosa Kernel Oil (And) Alumina |
| | | | (And) Methicone (And) |
| | | | Tocopheryl Acetate (And) |
| | | | Polyhydroxystearic |
| | | | Acid (And) Bisabolol |
| | | 100 | |

### Procedure:

1. Added Part 1 and heated to 85° C.
2. Pre-mixed Part 2 and added to Part 1

### 3. Cooled to 70-75° C.

4. Poured into molds at 70-75° C.

TABLE 2
Lipstick Formula with Phenyl Trimethicone @ 20 cSt

| Part | Ingredient | Percent | INCI |
|---|---|---|---|
| 1 | CPF-3300@20 cSt | 32.2 | Phenyl Trimethicone |
| 1 | Ozokerite Wax | 7.00 | Ozokerite |
| 1 | Koboguard HRPC | 7.00 | Hydrogenated Polyclopentadiene |
| | | | (and) Polyethylene (and) |
| | | | Copernicia Cerifera |
| | | | Wax (and) Tocopherol |
| 1 | Salacos WO-6 | 6.00 | Dipentaerythrityl Tri- |
| | | | Polyhydroxystearate |
| 1 | Carnuba Wax | 5.00 | Carnuba Wax |
| 1 | Microcrystalline | 4.00 | Microcrystalline |
| | Wax | | |
| 1 | Synthetic Beeswax | 4.00 | Beeswax |
| 2 | Cosmol 43V | 10.00 | Polyglyceryl-2 Triisostearate |
| 2 | INBP45R7C | 11.8 | Red 7 Lake (and) Isononyl |
| | | | Isononanoate (and) Isopropyl |
| | | | Myristate (and) Stearalkonium |
| | | | Hectorite (and) Isopropyl Titanium |
| | | | Triisostearate (and) Propylene |
| | | | Carbonate (and) |
| | | | Polyhydroxystearic Acid |
| 2 | KTZ Aruban Coral | 5.00 | Mica (and) Titanium Dioxide (and) |
| | | | Iron Oxides (C. I. 77491) |
| 2 | Kobopearl Perpetual | 3.00 | Synthetic Fluorphlogopite (and) |
| | RedGold | | Silica (and) Titanium Dioxide |
| 2 | TNP50T7-ATB | 4.00 | C12-15 Alkyl Benzoate (And) |
| | | | Titanium Dioxide (And) Argania |
| | | | Spinosa Kernel Oil (And) Alumina |
| | | | (And) Methicone (And) |
| | | | Tocopheryl Acetate (And) |
| | | | Polyhydroxystearic Acid |
| | | | (And) Bisabolol |
| | | 100 | |

### Procedure:

1. Added Part 1 and heated to 85° C.
2. Pre-mixed Part 2 and added to Part 1

### 3. Cooled to 70-75° C.

4. Poured into molds at 70-75° C.

TABLE 3
Lipstick Formula with Phenyl Trimethicone @ 40 cSt

| Part | Ingredient | Percent | INCI |
|---|---|---|---|
| 1 | CPF- | 32.2 | Phenyl Trimethicone |
| | 3300@40 cSt | | |
| 1 | Ozokerite Wax | 7.00 | Ozokerite |
| 1 | Koboguard | 7.00 | Hydrogenated Polyclopentadiene |
| | HRPC | | (and) Polyethylene |
| | | | (and) Copernicia Cerifera |
| | | | Wax (and) Tocopherol |
| 1 | Salacos WO-6 | 6.00 | Dipentaerythrityl Tri- |
| | | | Polyhydroxystearate |
| 1 | Carnuba Wax | 5.00 | Carnuba Wax |
| 1 | Microcrystalline | 4.00 | Microcrystalline |
| | Wax | | |
| 1 | Synthetic | 4.00 | Beeswax |
| | Beeswax | | |
| 2 | Cosmol 43V | 10.00 | Polyglyceryl-2 Triisostearate |
| 2 | INBP45R7C | 11.8 | Red 7 Lake (and) Isononyl |
| | | | Isononanoate (and) Isopropyl |
| | | | Myristate (and) Stearalkonium |
| | | | Hectorite (and) Isopropyl |
| | | | Titanium Triisostearate (and) |
| | | | Propylene Carbonate (and) |
| | | | Polyhydroxystearic Acid |
| 2 | KTZ Aruban | 5.00 | Mica (and) Titanium Dioxide (and) |
| | Coral | | Iron Oxides (C. I. 77491) |
| 2 | Kobopearl | 3.00 | Synthetic Fluorphlogopite (and) |
| | Perpetual | | Silica (and) Titanium Dioxide |
| | RedGold | | |
| 2 | TNP50T7-ATB | 4.00 | C12-15 Alkyl Benzoate (And) |
| | | | Titanium Dioxide (And) Argania |
| | | | Spinosa Kernel Oil (And) Alumina |
| | | | (And) Methicone (And) Tocopheryl |
| | | | Acetate (And) Polyhydroxystearic |
| | | | Acid (And) Bisabolol |
| | | 100 | |

### Procedure:

1. Added Part 1 and heated to 85° C.
2. Pre-mixed Part 2 and added to Part 1

### 3. Cooled to 70-75° C.

4. Poured into molds at 70-75° C.

### 3-Day Comparison Study on Lipstick Formulas Containing Phenyl Trimethicone @ 10 cSt and Phenyl Trimethicone @ 20 cSt

A 3-day lipstick study was conducted on 10 volunteer panelists using the following protocol:
Each volunteer wore 3 different Red lipstick products for 8 hours/day for 3 days.
The wear and transfer properties of these products were evaluated.
Use Instructions:
Prior to applying, each volunteer was asked to exfoliate and wash her lips with a scrub provided, and then to wipe with a Wet Wipe and allow her lips to dry prior to arrival at the Application Lab. After lipstick was applied on the lips for one (1) minute, the volunteer was given a glass plate to kiss. The product transfer was checked again at the end of the day.
Each volunteer was told to eat and drink as normal, but to refrain from wiping or washing her lips during the test period.

### Results Observed:

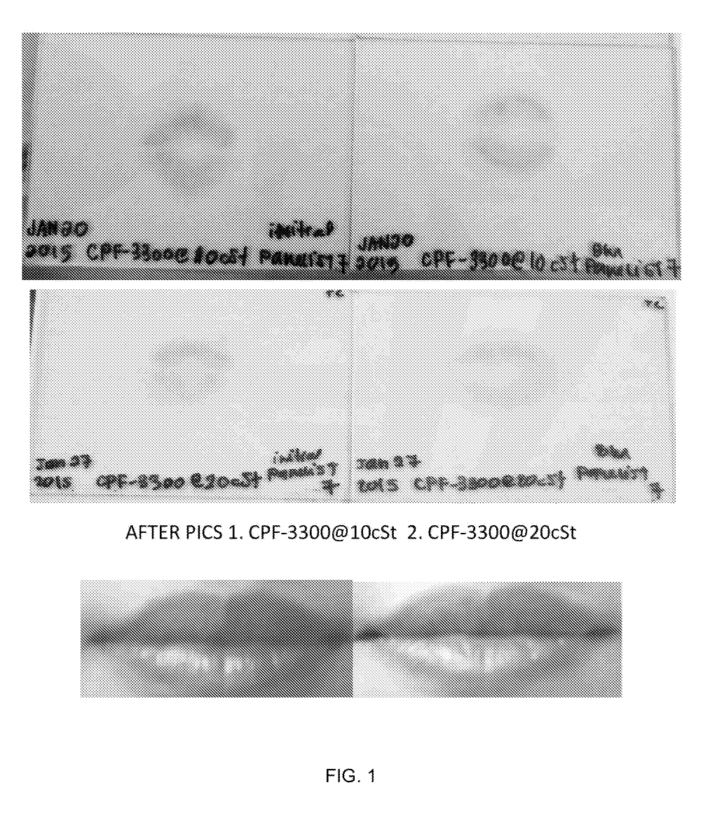

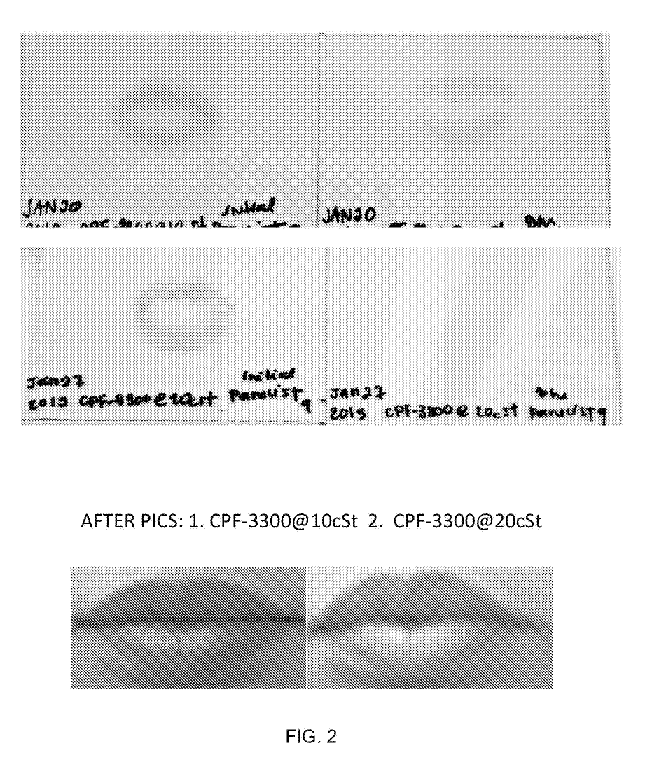

The comparison results of the lipstick or lipgloss formulas containing Phenyl Trimethicone @ 10 cSt or @20 cSt are illustrated in FIG. 1 and FIG. 2.

In FIG. 1, (top picture) glass plates on the left show initial transfer of lipsticks with either CPF-3300@10 cSt or CPF-3300@20 cSt test materials. Glass plates on the right show wear and transfer of lipstick with either CPF-3300@10 cSt or CPF-3300@20 cSt test materials after 8 hours of wear (bottom picture). The wear of lipstick with CPF-3300@10 cSt yielded better results compared to the lipstick containing CPF-3300@20 cSt as shown by both glass plates and lip pictures.

In FIG. 2, (top picture) glass plates on the left show initial transfer of lipsticks with either CPF-3300@10 cSt or CPF-3300@20 cSt test materials. Glass plates on the right show wear and transfer of lipstick with either CPF-3300@ 10 cSt or CPF-3300@20 cSt test materials after 8 hours of wear (bottom picture). The wear of lipstick with CPF-3300@10 cSt yielded better results compared to the lipstick containing CPF-3300@20 cSt as shown by both glass plates and lip pictures.

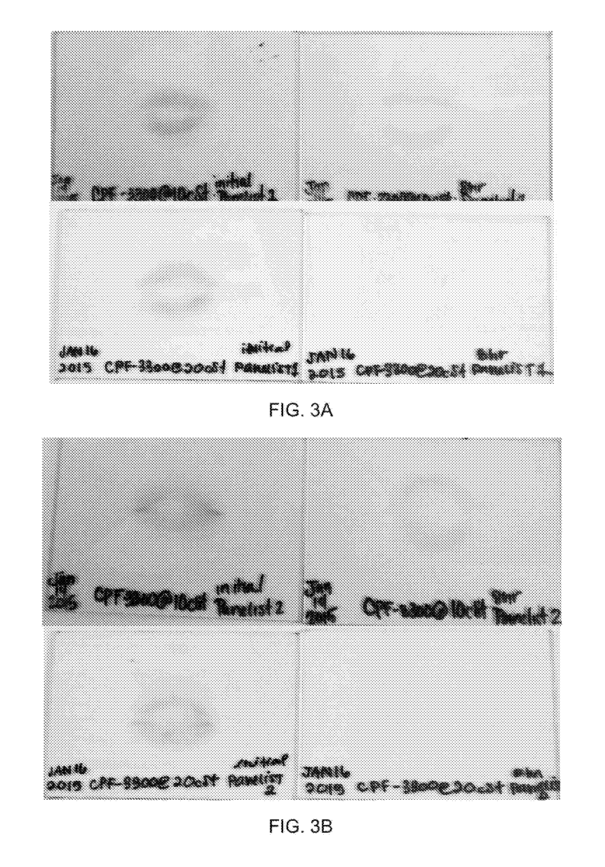

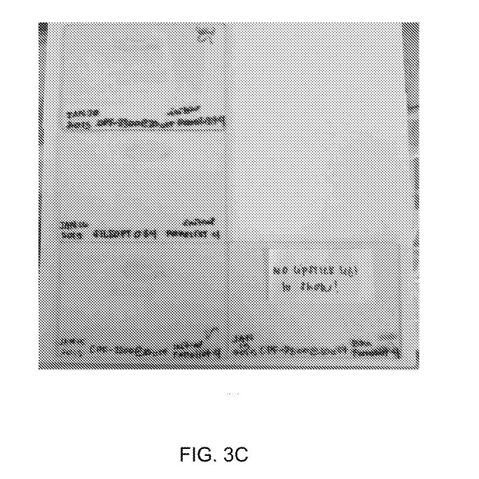

The representative results of the 3-day study on the above two lipstick formulas from Table 1 and Table 2 used on three panelists are also shown in FIGS. 3(A)-3(C), respectively. The results of this in-vivo transfer resistance test with lipstick demonstrate that the initial transfer yielded less product transferred to the glass plates in the formula containing Phenyl Trimethicone @ 10 cSt than the formula containing Phenyl Trimethicone @ 20 cSt.

### Example 2

Three lipgloss formulas containing Phenyl Trimethicone @ 10 cSt, Phenyl Trimethicone @ 20 cSt, and Phenyl Trimethicone @ 40 cSt with all other components kept the same, are described in Table 4, Table 5, and Table 6, respectively. They were prepared using the identical procedure as provided below.

TABLE 4
Lipgloss Formula with Phenyl Trimethicone @ 10 cSt

| Part | Percent | KOBO_Name | INCI Name |
|---|---|---|---|
| 1 | 42.42 | Panalene ® H-300E | Hydrogenated Polyisobutene |
| 1 | 40 | COSMOL ™ 222 | Diisostearyl Malate |
| 1 | 10 | CPF-3300 @10 cSt | Phenyl Trimethicone |
| 1 | 8 | SALACOS ® 5418V | Pentaerythrityl Tetraisostearate |
| 1 | 7.5 | Moonshine ® | Calcium Aluminum Borosilicate |
| | | Colour Travel | (And) Silica (And) Titanium |
| | | B-GR-G Shimmer | Dioxide (And) Tin Oxide |
| 1 | 2 | NOMCORT ® HK-G | Glyceryl Behenate/Eicosadioate |
| 1 | 0.08 | SW40R7C | Synthetic Wax (And) Red 7 |
| | | | Lake (And) Isopropyl Titanium |
| | | | Triisostearate |

### Procedure:

1. Mixed all ingredients.
2. Heated to 80° C.-85° C. with propeller mixing.
3. Cooled to 70° C. and poured into components.

TABLE 5
Lipgloss Formula with Phenyl Trimethicone @ 20 cSt

| Part | Percent | KOBO_Name | INCI Name |
|---|---|---|---|
| 1 | 42.42 | Panalene ® H-300E | Hydrogenated Polyisobutene |
| 1 | 40 | COSMOL ™ 222 | Diisostearyl Malate |
| 1 | 10 | CPF-3300 @20 cSt | Phenyl Trimethicone |
| 1 | 8 | SALACOS ® 5418V | Pentaerythrityl |
| | | | Tetraisostearate |
| 1 | 7.5 | Moonshine ® | Calcium Aluminum |
| | | Colour Travel B-GR-G | Borosilicate (And) Silica |
| | | Shimmer | (And) Titanium Dioxide |
| | | | (And) Tin Oxide |
| 1 | 2 | NOMCORT ® HK-G | Glyceryl |
| | | | Behenate/Eicosadioate |
| 1 | 0.08 | SW40R7C | Synthetic Wax (And) Red 7 |
| | | | Lake (And) Isopropyl |
| | | | Titanium Triisostearate |

### Procedure:

1. Mixed all ingredients.
2. Heated to 80° C.-85° C. with propeller mixing.
3. Cooled to 70° C. and poured into components.

TABLE 6
Lipgloss Formula with Phenyl Trimethicone @ 40 cSt

| Part | Percent | KOBO_Name | INCI Name |
|---|---|---|---|
| 1 | 42.42 | Panalene ® H-300E | Hydrogenated |
| | | | Polyisobutene |
| 1 | 40 | COSMOL ™ 222 | Diisostearyl Malate |
| 1 | 10 | CPF-3300 @40 cSt | Phenyl Trimethicone |
| 1 | 8 | SALACOS ® 5418V | Pentaerythrityl |
| | | | Tetraisostearate |
| 1 | 7.5 | Moonshine ® Colour Travel | Calcium Aluminum |
| | | B-GR-G Shimmer | Borosilicate (And) |
| | | | Silica (And) Titanium |
| | | | Dioxide (And) Tin |
| | | | Oxide |
| 1 | 2 | NOMCORT ® HK-G | Glyceryl |
| | | | Behenate/Eicosadioate |
| 1 | 0.08 | SW40R7C | Synthetic Wax (And) |
| | | | Red 7 Lake (And) |
| | | | Isopropyl Titanium |
| | | | Triisostearate |

### Procedure:

1. Mixed all ingredients.
2. Heated to 80° C.-85° C. with propeller mixing.
3. Cooled to 70° C. and poured into components.

### Example 3

Three mascara formulas containing Phenyl Trimethicone @ 10 cSt, Phenyl Trimethicone @ 20 cSt, and Phenyl Trimethicone @ 40 cSt with all other components kept the same, are described in Table 7, Table 8, and Table 9, respectively. They were prepared using the identical procedure as provided below.

TABLE 7
Mascara Formula with Phenyl Trimethicone @ 10 cSt

| Part | Ingredient | Percent | INCI |
|---|---|---|---|
| 1 | Mascon | 40.0 | Water (And) Iron Oxides (C. I. 77499) |
| | LT-100 | | (And) Polyethylene (And) |
| | | | Acrylates/Ethylhexyl Acrylate |
| | | | Copolymer (And) Copernicia Cerifera |
| | | | (Carnauba) Wax (And) Polymethyl |
| | | | Methacrylate (And) Alcohol (And) |
| | | | Lithium Magnesium Sodium Silicate |
| | | | (And) Nylon-6 (And) Caprylyl Glycol |
| | | | (And) Xanthan Gum (And) |
| | | | Triethanolamine (And) Lecithin (And) |
| | | | Sorbic Acid (And) Dimethicone (And) |
| | | | Sorbitan Stearate (And) PEG-40 |
| | | | Stearate (And) Ammonium |
| | | | Polyacrylate |
| 1 | CPF- | 10.0 | Phenyl Trimethicone |
| | 3300@10 cSt | | |
| 1 | Nomcort | 6.0 | Hydrogenated Polydecene |
| | HP-30 | | |
| 2 | Deionized | 37.5 | Water |
| | Water | | |
| 2 | PVP K-90 | 2.0 | Polyvinylpyrrolidone |
| 2 | Optiphen Plus | 0.40 | Phenoxyethanol (And) Caprylyl Glycol |
| | | | (And) Sorbic Acid |
| 3 | Glycerin | 3.0 | Glycerin |
| 3 | Natrosol | 0.60 | Hydroxyethylcellulose |
| | 250HHR CS | | |
| 3 | Aquakeep | 0.50 | Sodium Acrylates Crosspolymer-2 |
| | 10SH-NFC | | |
| | | 100 | |

### Procedure:

1. Added Part 1, and homogenized until fully homogenous
2. Added Part 2, added to Part 1 using side sweeping motion, and mixed for 15 minutes
3. Added Part 3, added to Parts 1 & 2, and mixed until fully homogenous

TABLE 8
Mascara Formula with Phenyl Trimethicone @ 20 cSt

| Part | Ingredient | Percent | INCI |
|---|---|---|---|
| 1 | Mascon | 40.0 | Water (And) Iron Oxides (C. I. 77499) |
| | LT-100 | | (And) Polyethylene (And) |
| | | | Acrylates/Ethylhexyl Acrylate |
| | | | Copolymer (And) Copernicia Cerifera |
| | | | (Carnauba) Wax (And) Polymethyl |
| | | | Methacrylate (And) Alcohol (And) |
| | | | Lithium Magnesium Sodium Silicate |
| | | | (And) Nylon-6 (And) Caprylyl Glycol |
| | | | (And) Xanthan Gum (And) |
| | | | Triethanolamine (And) Lecithin (And) |
| | | | Sorbic Acid (And) Dimethicone (And) |
| | | | Sorbitan Stearate (And) PEG-40 |
| | | | Stearate (And) Ammonium |
| | | | Polyacrylate |
| 1 | CPF- | 10.0 | Phenyl Trimethicone |
| | 3300@20 cSt | | |
| 1 | Nomcort | 6.0 | Hydrogenated Polydecene |
| | HP-30 | | |
| 2 | Deionized | 37.5 | Water |
| | Water | | |
| 2 | PVP K-90 | 2.0 | Polyvinylpyrrolidone |
| 2 | Optiphen Plus | 0.40 | Phenoxyethanol (And) Caprylyl Glycol |
| | | | (And) Sorbic Acid |
| 3 | Glycerin | 3.0 | Glycerin |
| 3 | Natrosol | 0.60 | Hydroxyethylcellulose |
| | 250HHR CS | | |
| 3 | Aquakeep | 0.50 | Sodium Acrylates Crosspolymer-2 |
| | 10SH-NFC | | |
| | | 100 | |

### Procedure:

1. Added Part 1, and homogenized until fully homogenous
2. Added Part 2, added to Part 1 using side sweeping motion, and mixed for 15 minutes
3. Added Part 3, added to Parts 1 & 2, and mixed until fully homogenous

TABLE 9
Mascara Formula with Phenyl Trimethicone @ 40 cSt

| Part | Ingredient | Percent | INCI |
|---|---|---|---|
| 1 | Mascon | 40.0 | Water (And) Iron Oxides (C. I. 77499) |
| | LT-100 | | (And) Polyethylene (And) |
| | | | Acrylates/Ethylhexyl Acrylate |
| | | | Copolymer (And) Copernicia Cerifera |
| | | | (Carnauba) Wax (And) Polymethyl |
| | | | Methacrylate (And) Alcohol (And) |
| | | | Lithium Magnesium Sodium Silicate |
| | | | (And) Nylon-6 (And) Caprylyl Glycol |
| | | | (And) Xanthan Gum (And) |
| | | | Triethanolamine (And) Lecithin (And) |
| | | | Sorbic Acid (And) Dimethicone (And) |
| | | | Sorbitan Stearate (And) PEG-40 |
| | | | Stearate (And) Ammonium |
| | | | Polyacrylate |
| 1 | CPF- | 10.0 | Phenyl Trimethicone |
| | 3300@40 cSt | | |
| 1 | Nomcort | 6.0 | Hydrogenated Polydecene |
| | HP-30 | | |
| 2 | Deionized | 37.5 | Water |
| | Water | | |
| 2 | PVP K-90 | 2.0 | Polyvinylpyrrolidone |
| 2 | Optiphen Plus | 0.40 | Phenoxyethanol (And) Caprylyl Glycol |
| | | | (And) Sorbic Acid |
| 3 | Glycerin | 3.0 | Glycerin |
| 3 | Natrosol | 0.60 | Hydroxyethylcellulose |
| | 250HHR CS | | |
| 3 | Aquakeep | 0.50 | Sodium Acrylates Crosspolymer-2 |
| | 10SH-NFC | | |
| | | 100 | |

### Procedure:

1. Added Part 1, and homogenized until fully homogenous
2. Added Part 2, added to Part 1 using side sweeping motion, and mixed for 15 minutes
3. Added Part 3, added to Parts 1 & 2, and mixed until fully homogenous

### Glossmeter Testing

### Objective:

To measure the specular reflection gloss using the Glossmeter of Phenyl Trimethicone (CPF-3300) at 10 cSt, 20 cSt, and 40 cSt. To measure the specular reflection gloss using the Glossmeter to compare lipstick, lipgloss and mascara formulas containing Phenyl Trimethicone (CPF-3300) at 10 cSt, 20 cSt, and 40 cSt.

### Glossmeter Results (GU=Gloss Units):

### Nusil Phenyl Trimethicone, CPF-3300 Materials

### 10 cSt: 53 GU @ 20°

### 20 cSt: 28 GU @ 20°

### 40 cSt: 24 GU @ 20°

### Lipstick Containing PT

### 10 cSt: 36 GU @ 85°

### 20 cSt: 32 GU @ 85°

### 40 cSt: 26 GU @ 85°

### Lipgloss Containing PT

### 10 cSt: 75 GU @ 20°

### 20 cSt: 74 GU @ 20°

### 40 cSt: 67 GU @ 20°

### Mascara Containing PT

### 10 cSt: 13.5 GU @ 20°

### 20 cSt: 11.5 GU @ 20°

### 40 cSt: 6.8 GU @ 20°

Since a higher gloss unit (GU) number indicates a higher gloss, from the comparison of various Phenyl Trimethicone-containing samples, the lipstick, lipgloss, and mascara formulas containing the Phenyl Trimethicone 10 cSt material yielded higher gloss units compared to test samples with the 20 and 40 cSt Phenyl Trimethicone.

### Example 4

Two comparative examples of eyeliner formulas containing Phenyl Trimethicone @ 10 cSt and Phenyl Trimethicone @ 20 cSt with all other components kept the same, are shown in Table 10 and Table 11, respectively. They were prepared using the identical procedure as provided below. These eyeliner formulas were subjected to water resistance and smudge-proof testing, and the results are listed in Table 12.

TABLE 10
Eyeliner formula (ALO04-75) with Phenyl Trimethicone @ 10 cSt

| Part | Ingredient | Percent | INCI |
|---|---|---|---|
| 1 | PMLVP20CB | 20.0 | Isododecane (and) Isohexadecane |
| | | | (and) Black 2 (and) Lecithin (and) |
| | | | Polyhydroxystearic Acid (and) |
| | | | Ethylene/Propylene/Styrene |
| | | | Copolymer (and) Butylene/ |
| | | | Ethylene/Styrene Copolymer |
| 1 | CPF- | 18.0 | Phenyl Trimethicone |
| | 3300@10 cSt | | |
| 1 | SR1000 | 15.0 | Trimethylsiloxysilicate |
| 1 | Ozokerite Wax | 9.0 | Ozokerite Wax |
| 1 | PM Wax 82 | 6.0 | Polyethylene (And) |
| | | | Microcrystalline Wax |
| 1 | Nomcort HK-G | 5.0 | Glyceryl Behenate/Eicosadioate |
| 1 | Sericite GMS- | 8.0 | Mica |
| | 4C | | |
| 1 | MSS-500W | 5.0 | Silica |
| 2 | Permethyl 99AD | 14.0 | Isododecane |
| | | 100 | |

### Procedure:

1. Combine Part 1 and heat to 90° C. Mix well under propeller until color is fully dispersed.
2. Cool to 80° C. and add Part 2.
3. Pour into components at 70-75° C.

TABLE 11
Eyeliner formula (ALO04-074) with Phenyl Trimethicone @ 20 cSt

| Part | Ingredient | Percent | INCI |
|---|---|---|---|
| 1 | PMLVP20CB | 20.0 | Isododecane (and) Isohexadecane (and) |
| | | | Black 2 (and) Lecithin (and) |
| | | | Polyhydroxystearic Acid (and) |
| | | | Ethylene/Propylene/Styrene Copolymer |
| | | | (and) Butylene/Ethylene/Styrene |
| | | | Copolymer |
| 1 | CPF- | 18.0 | Phenyl Trimethicone |
| | 3300@20 cSt | | |
| 1 | SR1000 | 15.0 | Trimethylsiloxysilicate |
| 1 | Ozokerite Wax | 9.0 | Ozokerite Wax |
| 1 | PM Wax 82 | 6.0 | Polyethylene (And) |
| | | | Microcrystalline Wax |
| 1 | Nomcort HK-G | 5.0 | Glyceryl Behenate/Eicosadioate |
| 1 | Sericite GMS- | 8.0 | Mica |
| | 4C | | |
| 1 | MSS-500W | 5.0 | Silica |
| 2 | Permethyl | 14.0 | Isododecane |
| | 99AD | | |
| | | 100 | |

### Procedure:

1. Combine Part 1 and heat to 90° C. Mix well under propeller until color is fully dispersed.
2. Cool to 80° C. and add Part 2.
3. Pour into components at 70-75° C.

TABLE 12
Results from water-resistance and smudge-proof testing of the eyeliner
formulas

| | Higher numbers are better | Higher numbers are |
|---|---|---|
| Products | Water-Resistance | Smudge-proof |
| AL004-074 | 97.43% | 73.10% |
| AL004-075 | 96.06% | 78.99% |

The results of this test demonstrate that Phenyl Trimethicone @ 10 cSt improved the smudge-proof performance of an anhydrous hot pour as compared to a corresponding formula using Phenyl Trimethicone @ 20 cSt.

### Example 5

A foundation formula containing Phenyl Trimethicone @ 10 cSt is listed in Table 13 below and was prepared according to the following procedure.

TABLE 13
Foundation Formula with Phenyl Trimethicone @ 10 cSt

| Part | Ingredients | Percent | INCI |
|---|---|---|---|
| 1 | SF1012 | 18.88 | Cyclopentasiloxane |
| 1 | TNP50T7 | 18.00 | C12-15 Alkyl Benzoate (and) Titanium |
| | | | Dioxide (and) Alumina (and) |
| | | | Polyhydroxystearic Acid (and) |
| | | | Methicone |
| 1 | FAS65UTB | 11.85 | Titanium Dioxide (and) |
| | | | Clyclopentasiloane (and) PEG/PPG- |
| | | | 18/18 Dimethicone (and) Isopropyl |
| | | | Titatnium Triisostearate (and) |
| | | | Triethoxysilylethyl |
| | | | Polydimethysiloxythyl Dimethicone |
| | | | (and) Disteardimonium Hectorite (and) |
| | | | Tocopheryl Acetate |
| 1 | CPF- | 3.50 | Phenyl Trimethicone |
| | 3300@10 cSt | | |
| 1 | FAS50YTB | 1.80 | Iron Oxide (C. I. 77492) (and) |
| | | | Clyclopentasiloane (and) PEG/PPG- |
| | | | 18/18 Dimethicone (and) Isopropyl |
| | | | Titatnium Triisostearate (and) |
| | | | Triethoxysilylethyl |
| | | | Polydimethysiloxythyl Dimethicone |
| | | | (and) Disteardimonium Hectorite (and) |
| | | | Tocopheryl Acetate |
| 1 | FAS65RTB | 0.30 | Iron Oxide (C. I. 77491 (and) |
| | | | Clyclopentasiloane (and) PEG/PPG- |
| | | | 18/18 Dimethicone (and) Isopropyl |
| | | | Titatnium Triisostearate (and) |
| | | | Triethoxysilylethyl |
| | | | Polydimethysiloxythyl Dimethicone |
| | | | (and) Disteardimonium Hectorite (and) |
| | | | Tocopheryl Acetate |
| 1 | FAS70BTB | 0.17 | Iron Oxide (C. I. 77499 (and) |
| | | | Clyclopentasiloane (and) PEG/PPG- |
| | | | 18/18 Dimethicone (and) Isopropyl |
| | | | Titatnium Triisostearate (and) |
| | | | Triethoxysilylethyl |
| | | | Polydimethysiloxythyl Dimethicone |
| | | | (and) Disteardimonium Hectorite (and) |
| | | | Tocopheryl Acetate |
| 1 | Propyl Paraben | 0.10 | Propylparaben |
| | NF | | |
| 2 | Water | 37.00 | |
| 2 | Butylene | 4.00 | Butylene Glycol |
| | Glycol | | |
| 2 | Sodium | 0.65 | Sodium Chloride |
| | Chloride | | |
| 2 | Polysorbate 20 | 0.40 | Polysorbate 20 |
| 2 | Methyl | 0.15 | Methylparaben |
| | Paraben NF | | |
| 2 | Trisodium | 0.10 | Trisodium EDTA |
| | EDTA | | |
| 2 | Allantoin | 0.10 | Allantoin |
| 3 | MSS-500W | 3.00 | Silica |
| | | 100 | |

### Procedure:

1. Homogenized all ingredients of Part 1 until fully uniform
2. Added Part 2 and fully mixed to the dispersed Part 1
3. Added Part 3 to Parts 1 and 2 slowly with homogenizing

The results of initial evaluation have shown that using Phenyl Trimethicone @ 10 cSt offered longer wear and more uniform product payoff than using Phenyl Trimethicone @ 20 cSt.

The foregoing examples or preferred embodiments are provided for illustration purpose and are not intended to limit the present invention. Numerous variations and combinations of the features set forth above can be utilized without departing from the present invention as set forth in the claims.

1. A cosmetic or personal care formulation comprising a low-viscosity phenyl trimethicone composition, wherein the low-viscosity phenyl trimethicone composition has a viscosity below 20 cSt.

2. The cosmetic or personal care formulation of claim 1, wherein said low-viscosity phenyl trimethicone composition has a viscosity in the range of 1 to 15 cSt.

3. The cosmetic or personal care formulation of claim 1, wherein said low-viscosity phenyl trimethicone composition is Phenyl Trimethicone @ 10 cSt, wherein use of Phenyl Trimethicone @ 10 cSt improves at least one property of the formulation in comparison with use of Phenyl Trimethicone @ 20 cSt.

4. The cosmetic or personal care formulation of claim 3, wherein said one property of the formulation is selected from the group consisting of shine, lasting effect upon wear, transfer resistance, smudge resistance, color development of the pigment in finished formula, and uniform product payoff.

5. The cosmetic or personal care formulation of claim 2, wherein the formulation is a lipstick, lipgloss, mascara, eyeliner, or foundation formula.

6. The cosmetic or personal care formulation of claim 5, wherein the lipstick formula comprises phenyl trimethicone @ 10 cSt in the amount of about 5-50% by weight.

7. (canceled)

8. (canceled)

9. The cosmetic or personal care formulation of claim 5, wherein the lipstick formula is substantially described in Table 1.

10. (canceled)

11. The cosmetic or personal care formulation of claim 5, wherein the lipgloss formula comprises phenyl trimethicone @ 10 cSt in the amount of about 1-50% by weight.

12. (canceled)

13. (canceled)

14. (canceled)

15. The cosmetic or personal care formulation of claim 5, wherein the lipgloss formula is substantially described in Table 4.

16. (canceled)

17. The cosmetic or personal care formulation of claim 5, wherein the mascara formula comprises phenyl trimethicone @ 10 cSt in the amount of about 1-50% by weight.

18. (canceled)

19. (canceled)

20. (canceled)

21. The cosmetic or personal care formulation of claim 5, wherein the mascara formula is substantially described in Table 7.

22. (canceled)

23. The cosmetic or personal care formulation of claim 5, wherein the eyeliner formula comprises phenyl trimethicone @ 10 cSt in the amount of about 1-50% by weight.

24. (canceled)

25. (canceled)

26. The cosmetic or personal care formulation of claim 5, wherein the eyeliner formula is substantially described in Table 10.

27. (canceled)

28. The cosmetic or personal care formulation of claim 5, wherein the foundation formula comprises phenyl trimethicone @ 10 cSt in the amount of about 1-70% by weight.

29. (canceled)

30. (canceled)

31. The cosmetic or personal care formulation of claim 5, wherein the foundation formula is substantially described in Table 13.

32. A method of preparing a cosmetic or personal care formulation, comprising including a low viscosity phenyl trimethicone as one of the ingredients of the formulation to enhance at least one property of the formulation selected from the group consisting of gloss, shine, lasting effect upon wear, transfer resistance, smudge resistance, color development of the pigment in finished formula, and uniform product payoff, wherein the low viscosity phenyl trimethicone has a viscosity below 20 cSt.

33. (canceled)

34. The method of claim 32, wherein said low viscosity phenyl trimethicone has a viscosity in the range of 1 to 15 cSt.

35. (canceled)

36. The method of claim 32, wherein said low viscosity phenyl trimethicone is Phenyl Trimethicone @ 10 cSt.

37. The method of claim 32, wherein said cosmetic or personal care formulation is a formulation used for lips, nails, skin, eyelashes, eyebrows, or hair.

38. The method of claim 37, wherein said cosmetic or personal care formulation is a formulation selected from the group consisting of lipstick, lipgloss, eyeliner, masacra, and foundation.

39. (canceled)

40. (canceled)
